(19) United States
(12) Patent Application Publication
(10) Pub. No.: US 2008/0182907 A1
(43) Pub. Date: Jul. 31, 2008

(54) External pharmaceutical composition containing tramadol

(75) Inventors: Kozo Takayama, Tokyo (JP); Yasuko Obata, Kawasaki (JP); Satoshi Nomoto, Yokohama (JP)

Correspondence Address: OLIFF & BERRIDGE, PLC, P.O. BOX 320850, ALEXANDRIA, VA, 22320-4850, US

(73) Assignee: NIPPON ZOKI PHARMACEUTICAL CO., LTD., Osaka-shi (JP)

(21) Appl. No.: 12/010,718

(22) Filed: Jan. 29, 2008

Related U.S. Application Data
(60) Provisional application No. 60/897,813, filed on Jan. 29, 2007.

(51) Int. Cl. A61K 31/135; A61P 29/00

(52) U.S. Cl. 514/646

(57) ABSTRACT

An analgesic composition for external application for increasing the percutaneous permeability of tramadol or a pharmaceutically acceptable salt thereof and for achieving a quick pharmacological effect. The analgesic composition includes tramadol or a pharmaceutically acceptable salt thereof, a menthol substance and a pyrrolidone compound.

This nonprovisional application claims the benefit of U.S. Provisional Application No. 60/897,813, filed Jan. 29, 2007.

### BACKGROUND

Disclosed herein is an external pharmaceutical composition for percutaneous application in which tramadol or a pharmaceutically acceptable salt thereof is contained as an effective ingredient.

Tramadol, trans-(±)-2-[(dimethylamino)methyl]-1-(3-methoxyphenyl)cyclohexanol, is a nonnarcotic synthetic analgesic being positioned between a strong narcotic analgesic with an indication of cancer pain, etc. and a nonsteroidal anti-inflammatory drug (NSAID) with an indication of mild pain such as headache and arthralgia. Tramadol has been receiving public attention as a drug having low frequency of onset of side effects to respiratory, circulatory and digestive systems and rarely causing resistance, physical dependence and abuse as compared with an opioid such as morphine of a narcotic analgesic. Tramadol has been found and developed by Grünenthal Ltd. (head office in Germany). A tramadol hydrochloride preparation was put into the market in 1977 in Germany as an oral preparation. Then it was sold in the form of injections and oral preparations in over 100 countries around the world.

Generally, as compared with an injection and an oral administration, a method of percutaneous administration is able to solve various problems by achieving a decrease in the number of doses due to persistence of effective blood level, avoidance of side effects due to lowering of the maximum drug concentration, elimination of the pain at injection and drip infusion, possibility of home care, avoidance of first-pass effect at an oral administration, improvement in compliance of patients and quality of life (QOL), and the like. However, depending upon the drug, there are some products which are barely absorbed percutaneously and so are scarcely formulated for external application. There is another problem that the external preparation is unable to provide a quick analgesic effect.

In Japan, a product of tramadol hydrochloride is currently sold in the injectable preparation alone. In other countries, although oral preparations are also sold, there is no product for external application. In the meanwhile, various research and development are now being carried out for tramadol preparations. For example, there are disclosures for a sustained-release oral preparation by adjusting the proportions of tramadol hydrochloride and a release modifying agent (Japanese Patent No. 3,045,924), and a preparation of tramadol available for nasal and inhalational applications (Japanese Patent No 3,152,437). The disclosure relating to an external preparation for percutaneous application is also disclosed, wherein the percutaneous permeability of tramadol is increased by the addition of menthol, or the like (Japanese Patent Laid-Open No. 2006/036,687).

### SUMMARY

An object of the present disclosure is thus to provide a novel analgesic composition for external application to increase the percutaneous permeation of tramadol or a pharmaceutically acceptable salt thereof as an effective ingredient, whereby a quick pharmacological effect can be achieved.

### BRIEF DESCRIPTION OF THE DRAWINGS

FIG. 1 is a result of in vitro skin permeability test showing the accumulated permeation amount of tramadol (TRA) when l-menthol and N-methyl-2-pyrrolidone (NMP) were added into a hydrogel containing tramadol hydrochloride as in Example 1.

FIG. 2 is a result of in vitro skin permeability test showing the lag time in percutaneous permeability of tramadol (TRA) when l-menthol and N-methyl-2-pyrrolidone (NMP) were added into a hydrogel containing tramadol hydrochloride as in Example 1.

### EMBODIMENTS

Under the circumstances where there has been almost no report for external preparation of tramadol, the present inventors have carried out intensive studies for percutaneous absorption of tramadol which is barely absorbed through the skin in order to achieve an excellent pharmacological effect as an external preparation. As a result, it has been found that, when a menthol substance such as menthol, O-ethylmenthol or limonene is combined with tramadol hydrochloride, enhancement of percutaneous absorption of tramadol is now achieved. Such enhancement has not been available in other absorption accelerators such as cholic acids. It has been further found that, when a pyrrolidone compound is additionally combined, time for the initiation of absorption of the drug (lag time) is shortened, whereby an analgesic effect is able to be achieved more quickly.

The external pharmaceutical composition in accordance with the present disclosure contains tramadol as an effective ingredient, having stronger analgesic action than NSAID, having lower frequency of onset of side effect and causing less resistance, physical dependence and abuse as compared with narcotic analgesics such as morphine. The composition has an excellent effect and is highly useful as an external analgesic agent that may be used safely for a long period.

The present disclosure relates to a new external pharmaceutical composition which contains tramadol or a pharmaceutically acceptable salt thereof as an effective ingredient, a menthol substance and a pyrrolidone compound.

Tramadol, which is an effective ingredient of the external pharmaceutical composition as disclosed herein, as used herein includes its salts, and there is no particular limitation for its use as long as it is a pharmaceutically acceptable salt. Examples thereof are inorganic acid salts such as hydrochloride, sulfate, nitrate, phosphate, hydrofluoride and hydrobromide and organic acid salts such as acetate, tartrate, lactate, citrate, fumarate, maleate, succinate, methanesulfonate, benzenesulfonate, toluenesulfonate, naphthalenesulfonate and camphorsulfonate. A preferred one is hydrochloride of tramadol (tramadol hydrochloride), which has been commercially available and widely used clinically as an analgesic agent. Stereoisomers, hydrates and solvates of tramadol are also included in tramadol, which is an effective ingredient of the present disclosure.

The menthol substance used in the external pharmaceutical composition in accordance with the present disclosure includes menthol, O-ethylmenthiol, limonene and stereoisomers thereof. Preferred examples are l-menthol, dl-menthol, O-ethylmenthol and d-limonene. Peppermint oil and peppermint solution in which menthol is a main component and lemon oil and orange oil in which limonene is a main component are also included in the menthol substance of the present disclosure.

The pyrrolidone compound used in the composition of the disclosure herein includes 2-pyrrolidone, N-methyl-2-pyrrolidone, 5-methyl-2-pyrrolidone, 1,5-dimethyl-2-pyrrolidone, 1-ethyl-2-pyrrolidone and 1-dodecyl-2-pyrrolidone. Among them, N-methyl-2-pyrrolidone (NMP) is preferred.

Percent by weight of tramadol or a pharmaceutically acceptable salt thereof used in the external pharmaceutical composition according to the present disclosure is from about 0.2 to about 10% by weight and, preferably, from about 0.5 to about 5% by weight when calculated as tramadol. With regard to the amount of the menthol substance used, it is from about 0.5 to about 20% by weight and, preferably, from about 1 to about 10% by weight for menthol and is from about 0.5 to about 30% by weight and, preferably, from about 0.5 to about 20% by weight for limonene and O-ethylmenthol, which are less irritative than menthol. The amount of the pyrrolidone compound is from about 1 to about 30% by weight and, preferably, from about 5 to about 20% by weight.

There is no particular limitation for the actual formulation of the external composition disclosed herein. It is possible to make into an external preparation such as liquid, cream, ointment, gel, lotion, spray, plaster and tape. In making into such pharmaceutical preparations, it is able to be manufactured by a common method, for example, mentioned in General Rules for Preparations in the Japanese Pharmacopoeia, etc., using additives and bases suitable for each formulation. It is also possible to make into a combination drug of tramadol and other effective drugs.

Preferred dosage forms are ointments and also gels such as a hydrogel, which is capable of being applied to plaster such as cataplasm and tape. The hydrogel can be prepared according to a common method where a viscous agent such as hydroxyethyl cellulose, hydroxypropyl cellulose, hydroxypropylmethyl cellulose and carboxyvinyl polymer is swelled with water to prepare a formulation base. To uniformly mix tramadol (main ingredient) and the above-mentioned additives in a preparation, it is possible to make into a pharmaceutical preparation using an appropriate solvent such as isopropanol and ethanol.

Preferred embodiments of the present disclosure are listed as follows.

(1) An external pharmaceutical composition which contains tramadol or a pharmaceutically acceptable salt thereof as an effective ingredient, a menthol substance and a pyrrolidone compound.

(2) The external pharmaceutical composition according to the above (1), wherein the effective ingredient is tramadol hydrochloride.

(3) The external pharmaceutical composition according to the above (1) or (2), wherein the pyrrolidone compound is N-methyl-2-pyrrolidone.

(4) The external pharmaceutical composition according to any of the above (1) to (3), wherein the menthol substance is l-menthol.

(5) The external pharmaceutical composition according to any of the above (1) to (3), wherein the menthol substance is d-limonene.

(6) The external pharmaceutical composition according to any of the above (1) to (3), wherein the menthol substance is O-ethylmenthol.

(7) The external pharmaceutical composition according to any of the above (1) to (6), wherein the composition is in a form of hydrogel.

(8) The external pharmaceutical composition according to any of the above (1) to (7), wherein isopropanol is used as a solvent.

(9) The external pharmaceutical composition according to any of the above (1) to (8), wherein the composition is an analgesic agent.

### EXAMPLES

The present disclosure is more specifically illustrated by way of the following Examples and Comparative Examples although the present disclosure is not limited thereto.

### Example 1

### Preparation of Hydrogel

Water (63% by weight) was added to 1% by weight of hydroxypropyl cellulose, 1% by weight of hydroxyethyl cellulose and 3% by weight of tramadol hydrochloride. Then, the mixture was allowed to stand for one night to swell the formulation base. Separately, 20% by weight of isopropanol (IPA), 2% by weight of l-menthol and 10% by weight of N-methyl-2-pyrrolidone (NMP) were mixed and gradually added to the base to prepare a homogeneous hydrogel. As to a Comparative Example, a composition without NMP was prepared, and depending on changes in the mixing amounts of the main ingredient and the additives, the amount of water was adjusted to make the total amount 100% by weight.

### Example 2

### In Vitro Skin Permeability Test

The full thickness of skin excised from abdomen of male hairless rats (8 weeks age) was cut out and mounted on a Franz-type diffusion cell (effective diffusion area: 2.0 cm²; receiver cell volume: 16.0 mL). The hydrogel (1.0 g) prepared in the above Example 1 was applied into a donor cell and the receiver cell was filled with a phosphate buffer of pH 7.2. Each 0.02 mL of the solution was collected every one hour from the receiver cell and tramadol was determined by a high performance liquid chromatography (HPLC). Thus, 0.2 mL of methanol in which 10 μg/mL of phenacetin was dissolved as an internal standard substance was added to 0.02 mL of the collected solution, stirred and injected to the HPLC. The HPLC was carried out under such a condition that a mobile phase was acetonitrile: phosphate buffer (0.01M, pH 3.0)=1:3, column was ODS (150 mm×4.6 mm), wavelength was 218 nm, flow rate was 1.0 mL/minute and column temperature was room temperature.

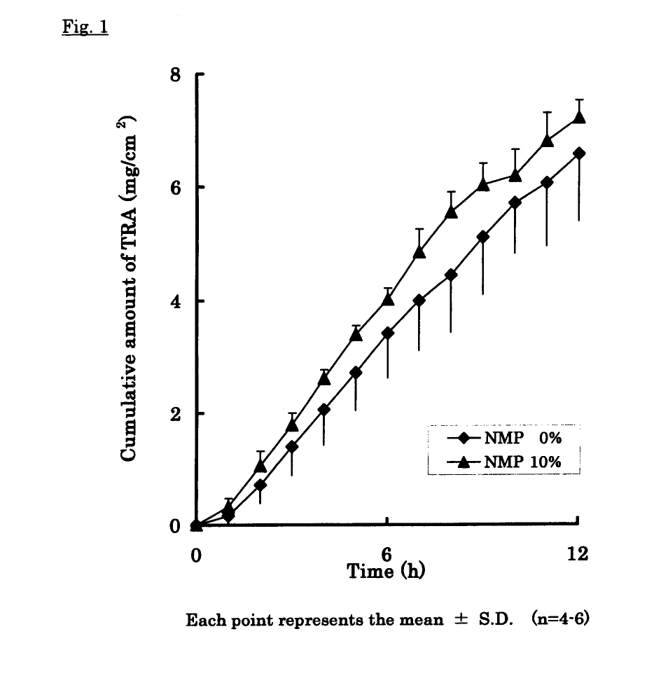

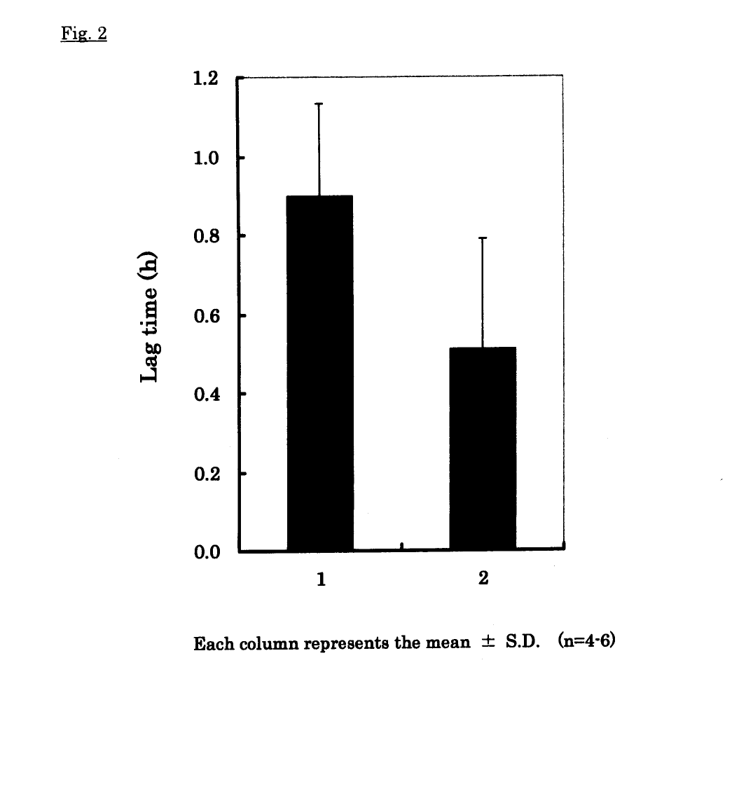

The permeating amount (mg/cm²) of tramadol through the skin and lag time (hour) were calculated from the above measured values. The results where the hydrogel added with N-methyl-2-pyrrolidone and l-menthol as a menthol substance was tested are shown in FIGS. 1 and 2. As apparent from the results, when both a menthol substance and N-methyl-2-pyrrolidone was combined, the percutaneous permeability of tramadol was increased and lag time was significantly shortened as compared with the case where only menthol was used.

### INDUSTRIAL APPLICABILITY

As shown from the result of the above pharmacological test, when both a menthol substance and a pyrrolidone compound were contained, the percutaneous permeability of tramadol was increased and time for initiation of permeation of the drug (lag time) was significantly shortened as compared with the case where only a menthol substance was contained. The composition of the present disclosure contains tramadol having stronger analgesic effect than non-steroidal anti-inflammatory drugs, showing lower frequency of onset of side effect and hardly resulting in resistance, physical dependence, abuse, etc., as compared with narcotic analgesics such as morphine. The composition of the present disclosure is highly useful as an external analgesic agent having an excellent percutaneous absorption and being expected to show quicker expression of analgesic effect.

What is claimed is:

1. An external pharmaceutical composition comprising an effective ingredient of tramadol or a pharmaceutically acceptable salt thereof in an effective pharmaceutical amount, a menthol substance and a pyrrolidone compound.

2. The external pharmaceutical composition according to claim 1, wherein the effective ingredient is tramadol hydrochloride.

3. The external pharmaceutical composition according to claim 1, wherein the pyrrolidone compound is N-methyl-2-pyrrolidone.

4. The external pharmaceutical composition according to claim 1, wherein the menthol substance is l-menthol.

5. The external pharmaceutical composition according to claim 1, wherein the menthol substance is d-limonene.

6. The external pharmaceutical composition according to claim 1, wherein the menthol substance is O-ethylmenthol.

7. The external pharmaceutical composition according to claim 1, wherein the composition is in a form of a hydrogel.

8. The external pharmaceutical composition according to claim 1, wherein isopropanol is used as a solvent.

9. The external pharmaceutical composition according to claim 1, wherein the composition is an analgesic agent.

10. An external pharmaceutical composition in a form of a hydrogel comprising an effective ingredient of tramadol hydrochloride in an effective pharmaceutical amount, 1-menthol and N-methyl-2-pyrrolidone.
